(19) United States
(12) Patent Application Publication
(10) Pub. No.: US 2026/0273219 A1
(43) Pub. Date: Sep. 17, 2026

(54) SYSTEM AND METHOD FOR ENHANCING LANGUAGE LEARNING THROUGH TARGETED TRANSCRANIAL ELECTRICAL STIMULATION

(71) Applicant: General Neuro Inc., Roslindale, MA (US)

(72) Inventors: Alec Sheffield, New Haven, CT (US); Luke Knoble, New Haven, CT (US)

(21) Appl. No.: 19/569,978

(22) Filed: Mar. 17, 2026

Related U.S. Application Data
(60) Provisional application No. 63/773,399, filed on Mar. 17, 2025.

(51) Int. Cl. A61M 21/00; A61N 1/04; A61N 1/36; G09B 5/02; G09B 5/06

(52) U.S. Cl. CPC A61M 21/00; A61N 1/0456; A61N 1/0476; A61N 1/0484; A61N 1/36025; G09B 5/02; A61M 2021/0072; A61M 2205/583; G09B 5/065

(57) ABSTRACT

The invention provides a system and method for enhancing language learning through transcranial electrical stimulation. The system combines a transcranial electrical stimulation headband with a language learning application utilizing spaced repetition algorithms. The application coordinates stimulation with vocabulary presentation, delivering stimulation synchronized with memory encoding events. The system assigns stimulation parameters to individual learning items, enabling characterization of dose-response relationships for a specific user from accumulated learning outcomes. The system distinguishes between short-term learning repetitions and long-term consolidation retrievals to isolate the memory consolidation signal for dose-response analysis. The system includes hardware verification of stimulation delivery through current measurement and comparison against commanded values. Various electrode designs may be employed, including arrays of conductive pins that allow for effective stimulation without requiring conductive gels, as well as extracephalic electrode configurations. Multiple dose selection strategies are supported, including predetermined schedules, randomized assignment, adaptive algorithmic optimization, and researcher-directed selection.

### CROSS-REFERENCE TO RELATED APPLICATIONS

This application claims priority to, and the benefit of, U.S. Provisional Application No. 63/773,399, filed Mar. 17, 2025, the content of which is incorporated by reference herein in its entirety.

### FIELD OF THE INVENTION

The present invention relates to systems and methods for enhancing language learning capabilities. More specifically, the invention relates to a combined hardware-software system that synchronizes transcranial electrical stimulation with language learning activities, characterizes individual dose-response relationships from learning outcomes, and optimizes stimulation parameters to improve vocabulary acquisition and retention.

### BACKGROUND OF THE INVENTION

Language acquisition is a complex cognitive process that engages multiple regions of the brain. Traditional language learning methods rely heavily on repetition, contextual learning, and memory techniques, but these approaches are limited by the brain's natural learning rate and retention capabilities.

It is well-established in neuroscience research that brain plasticity—the ability of neural networks to change through growth and reorganization—declines with age. This decline is particularly evident in language learning, where adults typically experience greater difficulty acquiring new languages compared to children. This phenomenon is often attributed to the closure of a “critical period” for language acquisition, after which the neural mechanisms that facilitate effortless language learning in children become less accessible.

Spaced repetition is a well-established learning technique based on the principle of the forgetting curve, first identified by Hermann Ebbinghaus in the late 19th century. Spaced repetition systems algorithmically determine the optimal timing for reviewing information by presenting items just as they are about to be forgotten, thereby strengthening the neural pathways associated with that memory. This evidence-based approach has been validated through decades of cognitive science research and is widely recognized as one of the most efficient methods for long-term knowledge retention. Nevertheless, even this scientifically-validated approach remains constrained by the natural limitations of adult neuroplasticity, suggesting that complementary approaches might further enhance learning outcomes.

Recent advances in neuroscience have demonstrated that targeted transcranial electrical stimulation (tES) can temporarily enhance neural plasticity and cognitive function. These techniques may potentially help overcome the age-related barriers to language learning by temporarily increasing plasticity in language-related brain regions. However, existing applications of tES have not been optimized specifically for language learning processes or integrated with language learning software in a synchronized manner.

Current tES devices often suffer from poor electrode contact, particularly in individuals with hair, requiring conductive gels or saline solutions. Additionally, these systems typically apply uniform stimulation parameters regardless of the specific cognitive task being performed, missing opportunities for task-specific optimization.

Furthermore, existing tES research predominantly employs between-subjects experimental designs to assess stimulation effects, which cannot capture individual variation in dose-response relationships. The optimal stimulation intensity may vary significantly between individuals due to differences in skull thickness, cortical anatomy, and baseline neural excitability. A system capable of characterizing individual dose-response relationships from that individual's own learning data would represent a significant advance over population-averaged approaches.

Beyond language acquisition for educational purposes, there exists a critical need for effective language rehabilitation technologies. Patients with acquired language disorders, such as post-stroke aphasia, traumatic brain injury, or neurodegenerative conditions, often face significant challenges in language processing, production, and comprehension. Transcranial electrical stimulation has shown promising results in small clinical studies for enhancing rehabilitation outcomes in these populations by potentially boosting neuroplasticity in damaged language networks. However, current approaches lack the precision targeting of specific language functions and the systematic integration with therapeutic exercises that would optimize recovery outcomes.

### SUMMARY OF THE INVENTION

The present invention, referred to as the NeuroLingo system, provides technology for enhancing language learning through transcranial electrical stimulation. At its core, the invention comprises methods and systems for applying targeted electrical stimulation to language-related brain regions in coordination with language learning activities. The system combines a specialized tES headband with a language learning application utilizing spaced repetition algorithms, synchronizing stimulation with learning processes to potentially enhance neuroplasticity during critical moments of language acquisition.

The invention includes a synchronized stimulation protocol wherein electrical stimulation is delivered during defined phases of the learning presentation. In a preferred embodiment, the learning presentation comprises a recall phase in which a target language item is displayed without stimulation, followed by an encoding phase in which the translation or answer is revealed with concurrent stimulation delivery. The stimulation comprises a controlled onset, sustained delivery at a target intensity, and controlled offset. This temporal coupling ensures stimulation coincides with the memory encoding event.

The system assigns stimulation parameters to individual learning items, and in a preferred embodiment, these parameters remain fixed across all subsequent reviews of that item. This enables each learning item to serve as an independent observation in a dose-response characterization. The system supports multiple strategies for selecting stimulation parameters, including predetermined schedules, randomized assignment, adaptive algorithmic methods, and researcher-directed selection, applied at the item level, session level, or day level.

The system characterizes the individual user's dose-response relationship from accumulated learning outcomes using statistical modeling techniques. An optimal stimulation intensity or set of parameters may be identified for the specific user based on this analysis.

A key innovation is the observation filtering methodology, wherein the system distinguishes between learning outcomes that reflect short-term or working memory and those that reflect long-term memory consolidation. Only consolidation-phase outcomes are included in the dose-response characterization, isolating the true memory consolidation signal from confounding short-term effects.

The hardware component includes a current measurement system that monitors the actual current delivered through the electrodes and compares it against commanded values, providing objective verification of stimulation delivery before each learning session.

### BRIEF DESCRIPTION OF THE DRAWINGS

FIG. 1 is a block diagram showing the overall architecture of the NeuroLingo system including both hardware and software components.

FIG. 2 is a top-down view of electrode montage configurations showing positioning over language-related brain regions.

FIG. 3 is a cross-sectional detail view of the spring-loaded pin electrode array design.

FIG. 4 is a diagram of the language learning application user interface.

FIG. 5 is a flow diagram illustrating the learning session protocol including device verification and card processing.

FIG. 6 is a diagram showing stimulation parameter assignment to vocabulary item subsets for dose-response testing.

FIG. 7 is a timing diagram illustrating the synchronized stimulation protocol, showing the temporal relationship between visual display, audio, and stimulation delivery during a single learning trial.

FIG. 8 is a diagram illustrating a dose-response model showing the relationship between stimulation intensity and predicted recall probability, with an exemplary Gaussian Process embodiment.

FIG. 9 is a diagram illustrating dose selection methods and assignment granularity options supported by the system.

FIG. 10 is a diagram showing the hardware current verification protocol including commanded versus measured current comparison.

FIG. 11 is a diagram showing the observation filtering methodology, distinguishing learning-phase repetitions from consolidation-phase retrievals based on memory state.

FIG. 12 is a diagram illustrating a smartphone-based visual guidance system for assisting users in positioning the headband over target brain regions.

FIG. 13 is a diagram illustrating a clinical rehabilitation system for adapting the stimulation platform for patients with acquired language disorders.

### DETAILED DESCRIPTION OF THE INVENTION

### System Overview

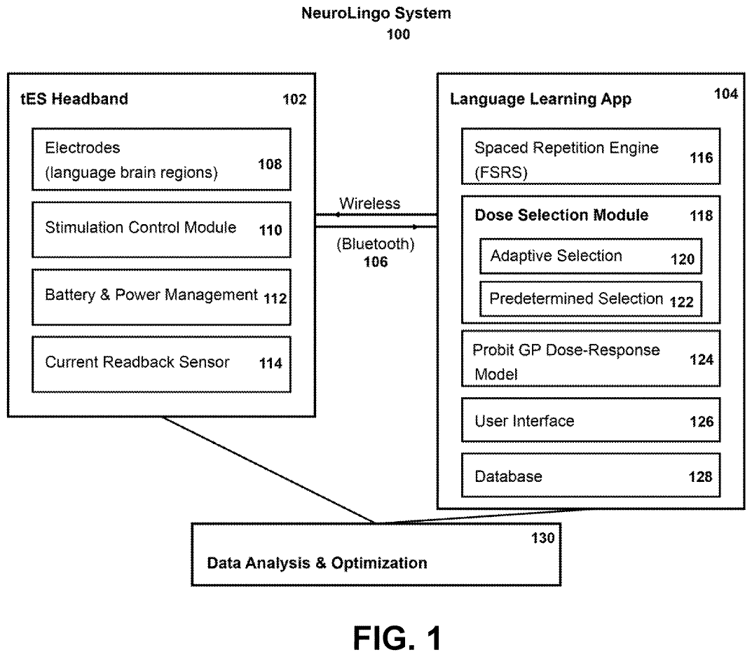

Referring to FIG. 1, the NeuroLingo system 100 comprises two main components: a transcranial electrical stimulation (tES) headband 102 and a language learning application 104. These components communicate via a wireless communication interface 106, such as Bluetooth, allowing the application 104 to trigger and modulate stimulation parameters based on the specific learning activity being performed.

The tES headband 102 includes electrodes 108 positioned to target language-related brain regions, a stimulation control module 110 configured to control stimulation parameters including current intensity, waveform, and duration, a battery and power management system 112, and a current readback sensor 114 configured to measure the actual current delivered through the electrodes for verification purposes.

The language learning application 104 includes a spaced repetition engine 116 configured to schedule learning item reviews based on predicted memory stability, a dose selection module 118 configured to assign stimulation parameters to learning items using any of a variety of methods, a dose-response model 124 for characterizing the relationship between stimulation parameters and learning outcomes for a specific individual, a user interface 126, and a database 128 for storing learning item data, review history, and dose-response observations. The dose selection module 118 may operate in an adaptive selection mode 120, wherein parameters are updated based on accumulated observations, or a predetermined selection mode 122, wherein parameters are assigned according to a fixed schedule such as evenly-spaced doses across the parameter range. The system further includes a data analysis and optimization module 130 for processing accumulated observations.

In one embodiment, the spaced repetition engine 116 implements the Free Spaced Repetition Scheduler (FSRS) algorithm, which uses a machine learning model to predict memory stability and optimize review intervals. In alternative embodiments, other spaced repetition algorithms such as SM-2, Leitner systems, or custom scheduling algorithms may be employed. The spaced repetition engine 116 may be configured with a target retention rate, for example 0.9, meaning items are scheduled for review when the predicted probability of correct recall drops to the target level.

### tES Headband

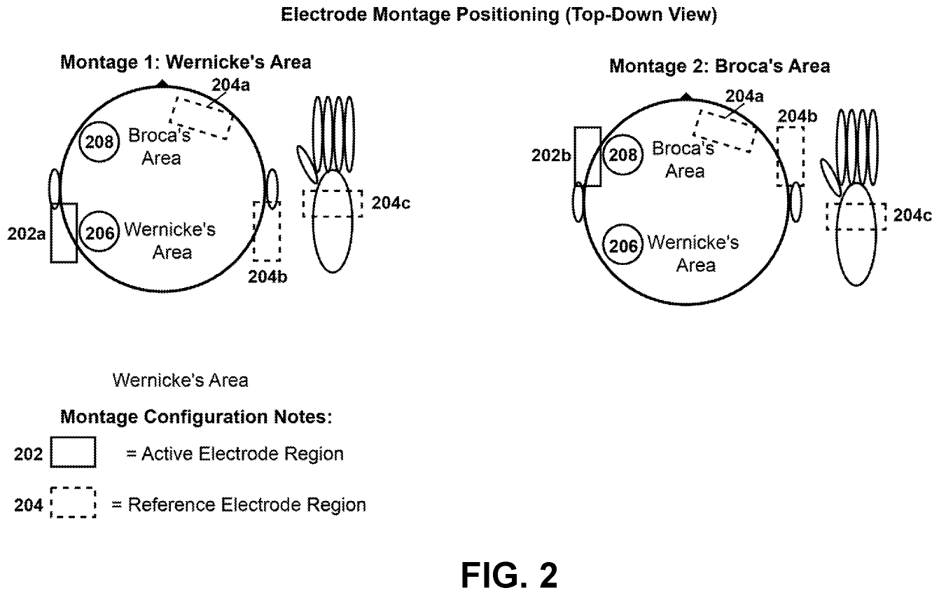

Referring to FIG. 2, the tES headband 102 is designed for comfort during extended learning sessions while maintaining precise electrode positioning over target brain regions. The headband incorporates electrodes that can be selectively activated to target specific language-related brain regions including Wernicke's area 206, associated with language comprehension, and Broca's area 208, associated with language production, as well as other language-related regions such as the arcuate fasciculus and superior temporal gyrus.

The headband supports multiple electrode montage configurations. The active electrode region 202 may be positioned over different target brain areas depending on the desired stimulation target: active position 202a targets Wernicke's area 206 for language comprehension enhancement, while active position 202b targets Broca's area 208 for language production enhancement. The reference electrode region 204 may be positioned at any of several locations, including: a contralateral supraorbital position 204a above the eyebrow on the opposite side, a contralateral hemisphere position 204b mirroring the active electrode placement on the opposite side of the head, or an extracephalic wrist position 204c using a wristband electrode. The selection of reference electrode position may depend on the desired current path, the target brain region, or user comfort. Additional montage configurations may target other language-related regions or employ bilateral stimulation.

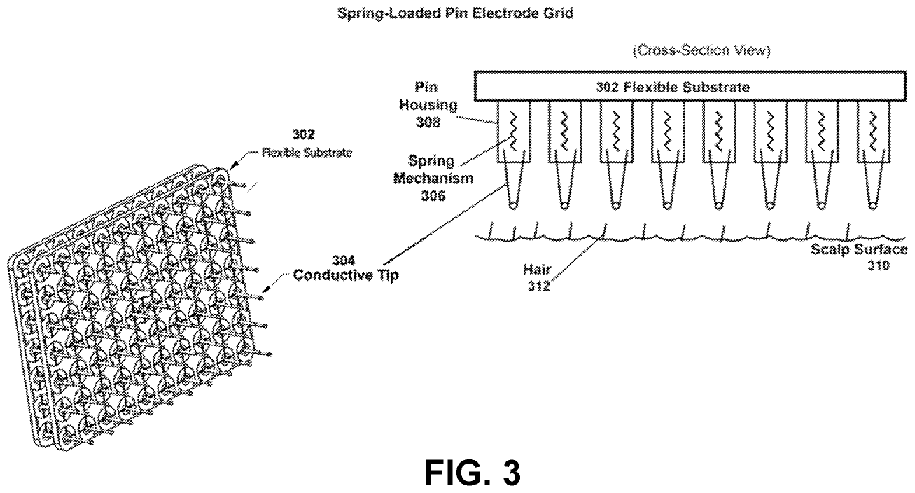

Referring to FIG. 3, the headband may incorporate various electrode designs including saline-soaked sponge electrodes, hydrogel-based conductive interfaces, dry electrode technologies, or arrays of conductive pins. In one embodiment, the electrode design features a grid of spring-loaded pins mounted on a flexible substrate 302. Each pin includes a conductive tip 304 for optimal electrical conductivity, a spring mechanism 306 allowing individual pins to adjust to the contour of the head, and a pin housing 308 within the substrate. The pins at varying extensions accommodate the scalp surface 310 and hair 312, enabling effective electrical contact through hair without requiring conductive gels or saline solutions.

The headband may include an ear reference tab configured to hook over the pinna of the user's ear, providing a consistent anatomical reference point for anterior-posterior positioning of the headband. The ear reference tab ensures repeatable electrode placement across sessions without requiring external measurement tools or imaging-based guidance, enabling the user to self-position the headband reliably.

The headband may also incorporate extracephalic electrodes implemented with a wristband design functioning as reference or return electrodes, or used to create current paths targeting deeper brain regions.

The stimulation control module 110 receives commands from the language learning application 104, controls stimulation parameters including current intensity within a configurable range (for example, 0 to 4 milliamps), waveform characteristics, and duration, monitors electrode contact quality and user safety parameters, and transmits current measurement data to the application for verification.

### Language Learning Application

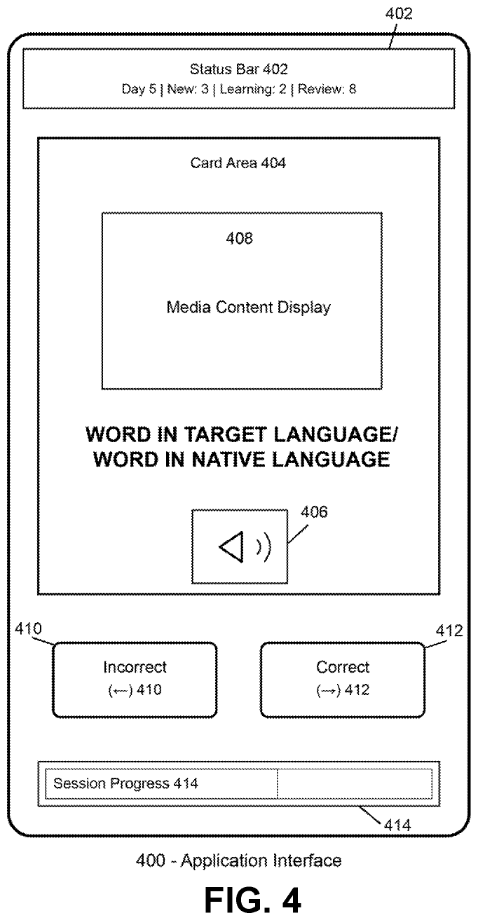

Referring to FIG. 4, the language learning application 104 provides a user interface 400 comprising a status bar 402 showing session information including current day, and counts of new, learning, and review items. A card area 404 serves as the main display region for presenting learning content. Within the card area, a media content display 408 presents visual or multimedia content associated with the learning item, such as images or video clips selected for emotional salience to aid in memory encoding and recall. An audio playback control 406 provides audio pronunciation or other auditory content. Response input mechanisms 410, 412 allow the user to indicate whether recall was incorrect 410 or correct 412. In a preferred embodiment, the response input is implemented as a swipe gesture, wherein a leftward swipe indicates incorrect recall and a rightward swipe indicates correct recall; in alternative embodiments, the response input may be implemented as discrete buttons. A session progress indicator 414 displays overall progress through the current learning session.

The application records learning outcomes using binary ratings (correct or incorrect) or multi-level ratings, reducing decision complexity for the user while providing sufficient signal for dose-response analysis. The spaced repetition engine 116 maintains learning items in states including at least: a learning state for recently introduced items undergoing initial repetitions, a review state for items that have graduated to spaced retrieval, and a relearning state for items that were previously learned but failed during a review. The distinction between these states is utilized by the observation filtering methodology described below.

### Methods of Operation

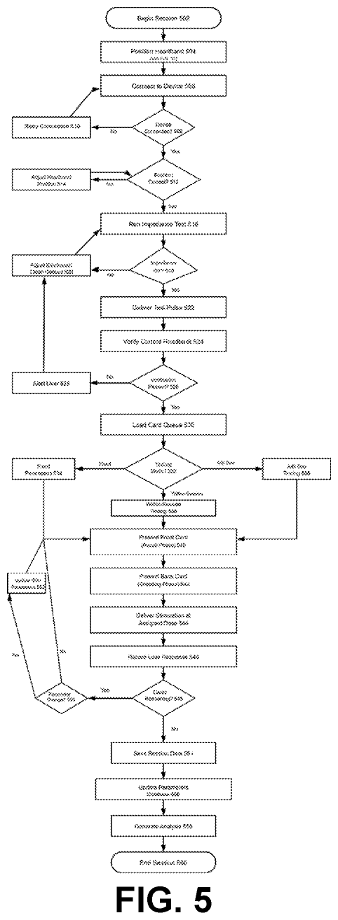

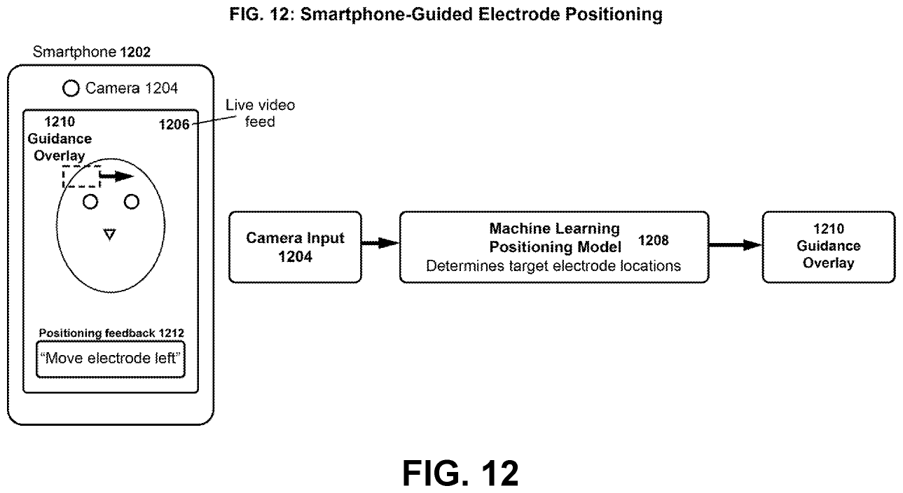

Referring to FIG. 5, the NeuroLingo system operates according to a session protocol. The user first positions the headband at step 504, optionally using the smartphone-based visual guidance feature described with reference to FIG. 12. The system then establishes a connection to the tES device at stepb 506. Upon successful connection at decision 508, the system verifies headband positioning at decision 512. The system then runs an impedance test at step 516 to confirm adequate electrode contact at decision 518, followed by a current verification procedure comprising a test pulse at step 522 and current readback verification at step 524. Upon successful verification at decision 526, the learning item queue is loaded at step 530, comprising new items, items in the learning state, items in the relearning state, and items scheduled for review. The user selects a testing mode at decision 532, which may include fixed parameters 534, A/B day testing 536, or within-session testing 538. For each item in the queue, the system presents a front card during a recall phase 540, followed by a back card during an encoding phase 542, delivers stimulation at the assigned dose 544, and records the user response 546. At decision 548, the system checks whether cards remain; if so, it optionally checks whether a within-session parameter change 550 is needed and updates stimulation parameters 552 accordingly before proceeding to the next card. When all cards are processed, the system saves session data 554, updates the parameters database 556, generates analysis 558, and ends the session 560.

For each item in the queue, a learning presentation is displayed comprising at least a recall phase and an encoding phase. During the encoding phase, stimulation is delivered at the parameters assigned to that item. The user's response is recorded and the spaced repetition schedule is updated. The system determines whether the review qualifies as a consolidation-phase observation based on the item's memory state; if so, the observation is recorded for dose-response analysis. The session continues until all items are processed.

### Item-Level Stimulation Parameter Assignment

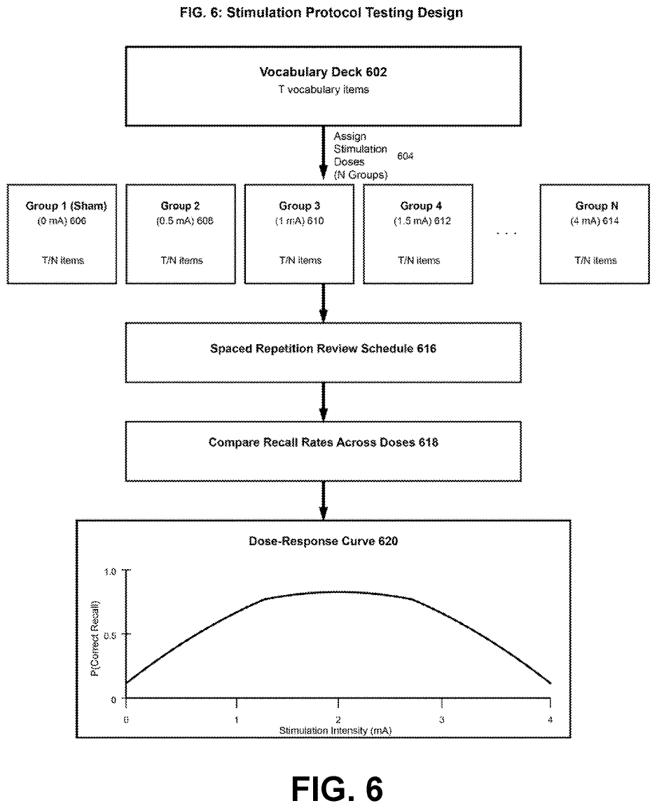

Referring to FIG. 6, the system assigns stimulation parameters to learning items to enable systematic evaluation of stimulation efficacy and characterization of dose-response relationships. A set of T learning items 602 is divided through a dose assignment process 604 into N groups that receive different stimulation parameters spanning the parameter range. The number of groups N and the specific dose levels assigned to each group may be determined by any of the dose selection methods described herein, including predetermined evenly-spaced values, randomized assignment, or adaptive algorithmic selection such as Bayesian optimization. In one exemplary arrangement, the groups include a sham condition at zero intensity 606 and various active stimulation levels 608, 610, 612, 614; however, any number of groups and dose levels may be used depending on the size of the parameter space and the number of available learning items.

In a preferred embodiment, stimulation parameters are assigned at the individual item level, such that each learning item receives a specific set of stimulation parameters at the time of its first introduction. These parameters may remain fixed across all subsequent reviews of that item, enabling each item to serve as an independent observation in a dose-response experiment. The item's learning outcomes across multiple spaced reviews provide repeated measurements at that item's assigned dose level.

In alternative embodiments, stimulation parameters may be assigned at the session level (all items within a session receive the same parameters), at the day level (alternating parameters across days in an A/B or randomized pattern), or at the block level (groups of items within a session share parameters). Item-level assignment is preferred because it maximizes the number of distinct dose-response observations collected per session.

### Synchronized Stimulation Protocol

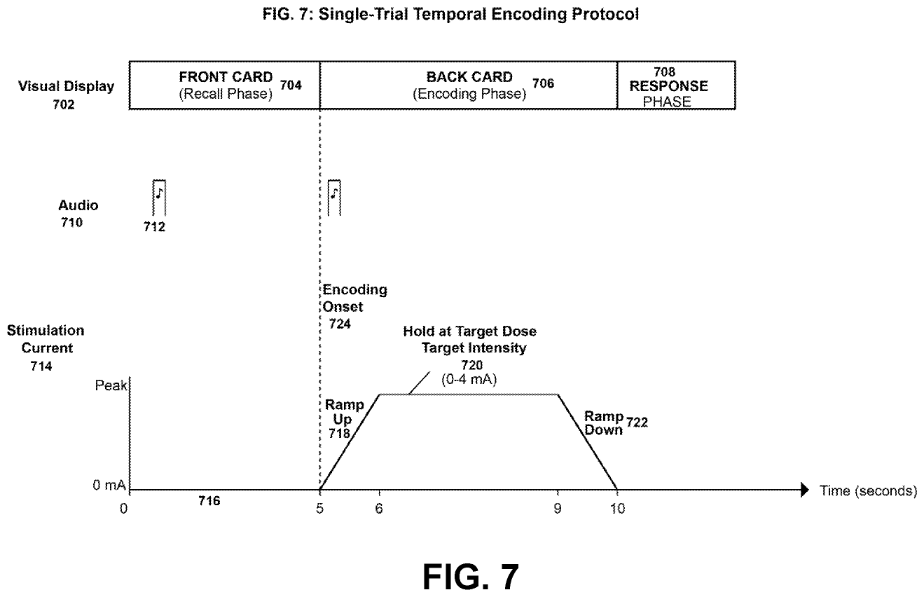

Referring to FIG. 7, the synchronized stimulation protocol defines the temporal relationship between learning content presentation and electrical stimulation delivery. This protocol represents a departure from conventional tES approaches that apply continuous stimulation over extended periods.

The learning presentation comprises at least two phases. The front card or recall phase 704 displays a prompt (for example, a word in the target language) without stimulation, during which the user attempts recall. The back card or encoding phase 706 reveals the answer (for example, the translation) with concurrent stimulation delivery. An optional response phase 708 follows, during which the user provides a response indicating recall success or failure.

The stimulation current is temporally locked to the encoding phase. At the encoding onset 724, the stimulation current begins a controlled ramp up 718 from zero to a target intensity. The current is then held at the target intensity during a hold period 720. The current then undergoes a controlled ramp down 722 from the target intensity back to zero. The ramp periods prevent abrupt current changes that could cause discomfort or artifact.

In one embodiment, the encoding phase is approximately 5 seconds, with a ramp-up period of approximately 1 second, a hold period of approximately 3 seconds, and a ramp-down period of approximately 1 second. In alternative embodiments, these durations may be adjusted based on the learning content type, the stimulation parameters, or the specific cognitive process being targeted. Stimulation may alternatively be delivered during the recall phase, during both phases, or at other defined time points relative to the learning event.

### Individual Dose-Response Characterization

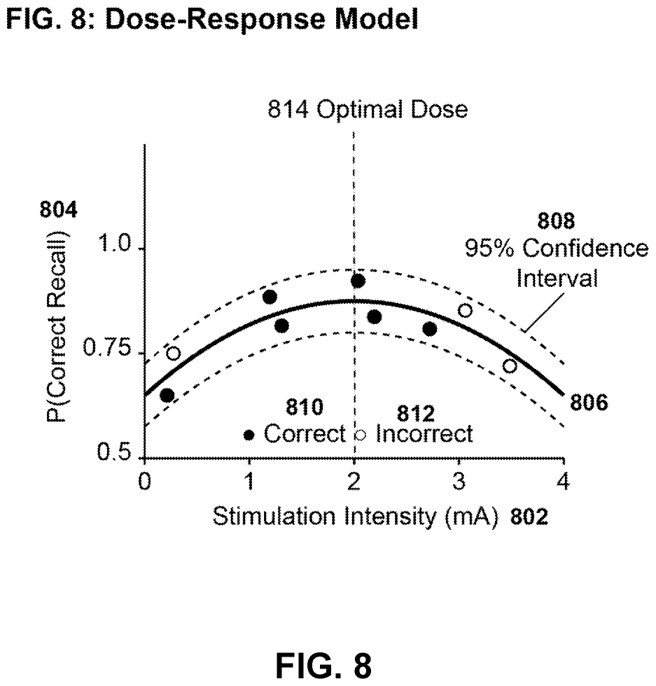

Referring to FIG. 8, the system characterizes the relationship between stimulation parameters and learning outcomes for a specific individual. Unlike between-subjects designs common in tES research, this approach collects sufficient data from a single user across many learning items and review sessions to estimate that individual's dose-response curve.

The dose-response model 124 takes as input the stimulation intensity and binary or multi-level learning outcome for each qualifying observation, and produces an estimate of the probability of successful recall as a function of stimulation intensity. The model may identify an optimal stimulation intensity 814 that maximizes predicted recall probability, which may then be used to configure stimulation for future learning sessions.

The dose-response relationship may be characterized using any suitable statistical or machine learning technique, including but not limited to: logistic regression, Gaussian Process models, Bayesian optimization, neural network classifiers, non-parametric methods such as kernel density estimation, binomial proportion analysis, or any combination thereof.

In one embodiment, the system employs a Probit Gaussian Process model, wherein a latent function f(dose) with a Gaussian Process prior is mapped through a probit link function to yield predicted recall probabilities as P(correct |dose)=Φ(f(dose)). The Gaussian Process uses a radial basis function (RBF) kernel to encode the assumption that the dose-response relationship varies smoothly across the stimulation range. Posterior inference is performed using Laplace approximation, and predictive distributions at new dose values are computed using standard GP prediction equations with numerically stable Cholesky factorization. This specific embodiment is particularly suited to the binary outcome, continuous-covariate structure of the dose-response problem.

### Dose Selection Methods

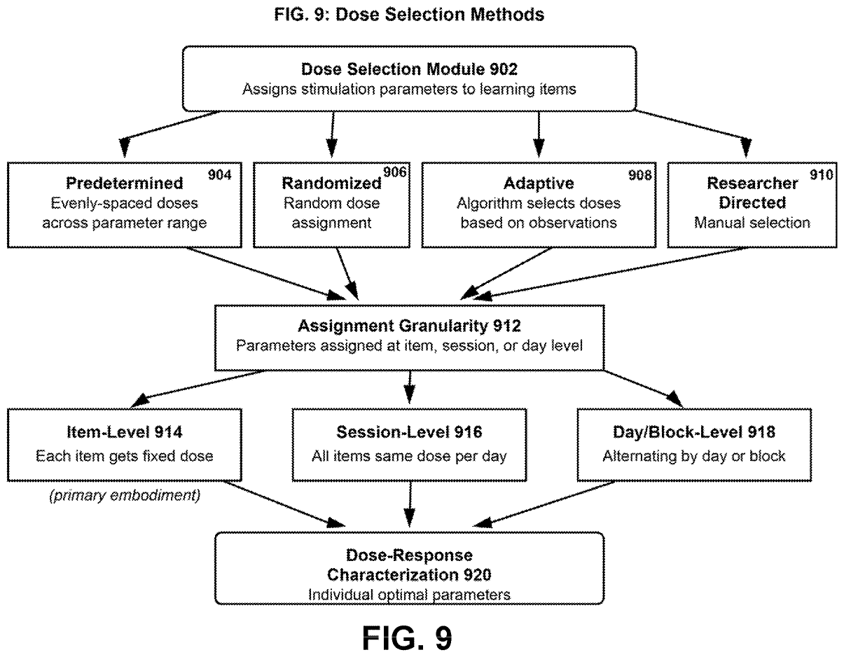

Referring to FIG. 9, the dose selection module 902 supports multiple strategies for assigning stimulation parameters to learning items. The selection of strategy depends on factors such as the size of the parameter space to be explored, the number of available learning items, the desired balance between exploration and exploitation, and whether the system is operating in a research or optimized-use mode.

Predetermined dose selection 904 assigns evenly-spaced doses across the parameter range, providing equal statistical power at all dose levels. This approach is preferred when the parameter space is small or when unconfounded estimation across the full range is desired. Randomized dose selection 906 assigns doses randomly from the parameter space. Adaptive dose selection 908 uses algorithmic methods that update dose assignments based on accumulated observations, concentrating sampling in informative regions of the parameter space. Researcher-directed selection 910 allows manual specification of doses based on domain knowledge or experimental requirements.

In one embodiment, adaptive dose selection employs Bayesian optimization with an Upper Confidence Bound (UCB) acquisition function that balances exploitation of promising parameter values with exploration of uncertain regions. In alternative embodiments, other adaptive strategies may be used, including Thompson sampling, expected improvement, gradient-based optimization, evolutionary algorithms, or multi-armed bandit approaches.

The assignment granularity 912 determines the level at which parameters are assigned. Item-level assignment 914, wherein each learning item receives a fixed set of parameters, is the primary embodiment as it maximizes the number of independent dose-response observations per session. Session-level 916 and day-level or block-level 918 assignment are alternative embodiments that may be used alone or in combination with item-level assignment.

The various dose selection methods and granularity levels may be used alone, in sequence, or in any combination. For example, an initial period of adaptive selection may be followed by predetermined selection, or different methods may be applied to different subsets of learning items within the same session.

### Hardware Current Verification

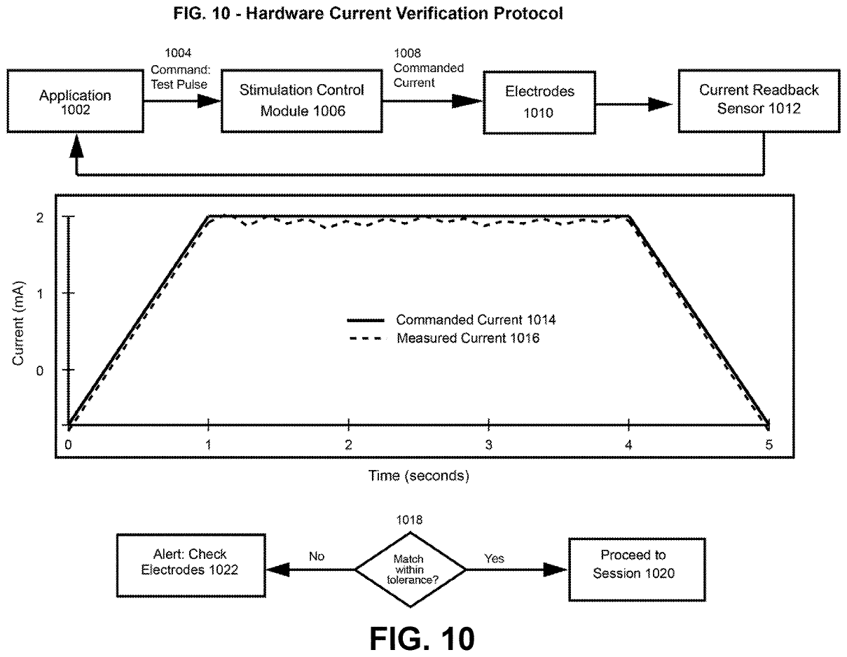

Referring to FIG. 10, the hardware current verification protocol ensures reliable stimulation delivery before each learning session. The application 1002 sends a command for a test pulse 1004 to the stimulation control module 1006. The commanded current 1008 is delivered through the electrodes 1010.

The current readback sensor 1012 measures the actual current flowing through the electrodes at a sufficient sampling rate to characterize the stimulation waveform. The commanded current waveform 1014 is compared against the measured current waveform 1016 to verify delivery accuracy. At decision point 1018, the system evaluates whether the commanded and measured currents correspond within acceptable tolerance. If verification passes, the session proceeds 1020. If verification fails, an alert is generated 1022 prompting the user to check electrode placement or contact quality. This objective verification provides quantitative confirmation that electrode impedance is adequate for reliable stimulation delivery.

### Observation Filtering Based on Memory Consolidation State

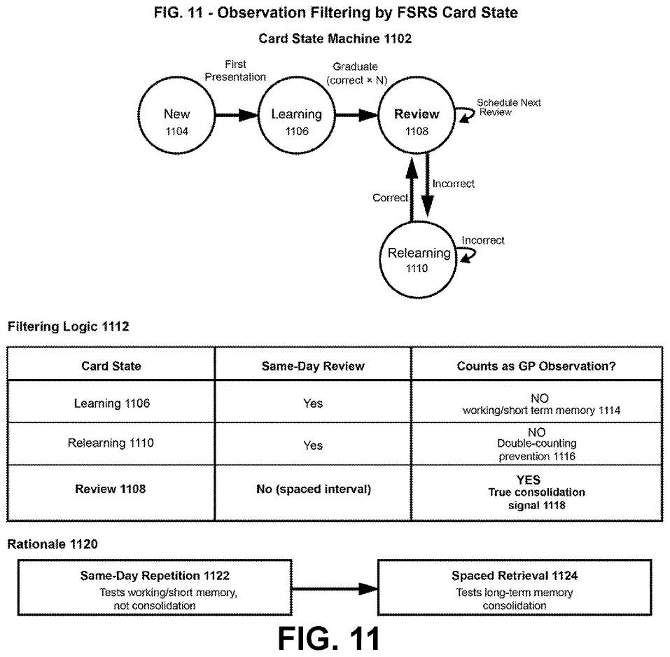

Referring to FIG. 11, the observation filtering methodology distinguishes between learning outcomes that reflect different memory processes. Not all assessments of learning item knowledge equally reflect the long-term memory consolidation that transcranial electrical stimulation is hypothesized to enhance. The system filters observations to include only those that meaningfully reflect consolidation-phase memory retrieval.

The system classifies each learning outcome based on the memory consolidation state of the item at the time of review. Short-term assessments—such as same-session repetitions during initial learning or re-learning after a failure—primarily reflect short-term or working memory rather than long-term consolidation. These outcomes are excluded from dose-response analysis. Only outcomes that reflect retrieval after a meaningful time interval, during which consolidation processes have had the opportunity to act, are included as dose-response observations.

In one embodiment using a spaced repetition system, the filtering is implemented based on card review states. Reviews occurring while a card is in a Learning state 1106 are excluded because they occur on the same day as introduction and therefore test short-term memory rather than consolidated recall. Reviews occurring while a card is in a Relearning state 1110 are excluded to prevent double-counting of a single failed review event. Only reviews occurring while a card is in a Review state 1108, which occur after a spaced interval, are counted as consolidation-phase observations 1118. In alternative embodiments, the filtering may be based on elapsed time since last presentation, number of intervening items, or any other indicator of whether the assessment reflects consolidated memory.

### Smartphone-guided Electrode Positioning

Referring to FIG. 12, the system may include a smartphone-based visual guidance feature for assisting users in positioning the headband. A smartphone 1202 includes a camera 1204 that captures a live video feed of the user's head. The camera input 1204 is provided to a machine learning positioning model 1208 that analyzes the captured image to determine the user's head geometry and identify target electrode positions relative to anatomical landmarks. The machine learning positioning model 1208 outputs a guidance overlay 1210 comprising visual reference points displayed on the smartphone display screen 1206, showing the user where to place the headband relative to their current head position. Positioning feedback 1212 provides real-time textual instructions (for example, “Move electrode left”) indicating adjustments needed to achieve correct headband placement.

### Stimulation Parameter Variations

The system supports multiple stimulation waveform types including direct current (DC) stimulation, alternating current (AC) at various frequencies, random noise stimulation, and arbitrary waveform combinations thereof (such as DC+noise, DC+AC, pulsed patterns). Current intensity is adjustable within a configurable range. Temporal patterns, electrode montages, and stimulation durations may all be varied as part of the dose-response characterization.

### Testing Methodologies

The system supports multiple testing methodologies for evaluating stimulation efficacy, including: A/B testing with alternating stimulation protocols by day; randomized testing with randomly assigned protocols across sessions; within-session testing with different protocols applied to different learning items within the same session; item-level dose variation as described above; and longitudinal analysis tracking retention rates across different stimulation conditions over extended periods.

### Sham Stimulation Mode

For efficacy testing and control conditions, the system includes a sham stimulation mode. Current is applied only briefly at the beginning and end of the stimulation period with ramp-up and ramp-down periods creating the sensation of stimulation. No current is delivered during the primary learning period. The user is not informed whether they are receiving active or sham stimulation.

### Therapeutic Applications for Language Rehabilitation

The NeuroLingo system may be adapted for therapeutic applications in language rehabilitation, particularly for patients with acquired language disorders such as aphasia. The stimulation protocols may be modified to target specific damaged language networks based on individual patient assessment. The language learning content may be adapted to focus on rehabilitation exercises, including naming tasks, comprehension activities, and speech production exercises. The system may include progress tracking specific to clinical outcomes, potentially generating reports for healthcare providers. Stimulation parameters may be optimized for rehabilitation contexts, potentially using different intensity levels or stimulation durations than those used for educational purposes. The application may incorporate standardized assessment tools to measure and track clinical improvement. User interfaces may be simplified and adapted for individuals with cognitive or perceptual impairments.

The system's ability to characterize individual dose-response relationships may be particularly valuable in rehabilitation settings, where optimal parameters may vary significantly between patients based on lesion location, severity, and time post-injury.

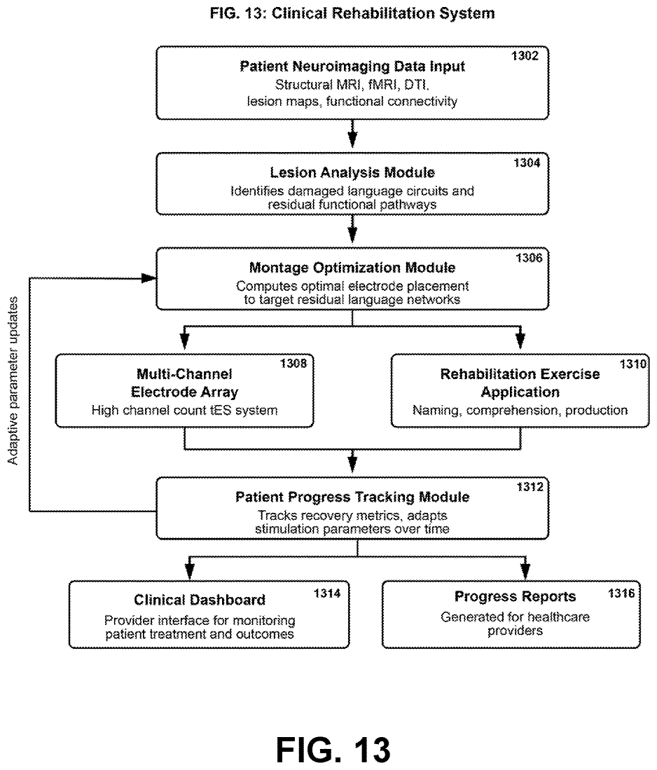

Referring to FIG. 13, a clinical rehabilitation embodiment of the system receives patient neuroimaging data input 1302, which may include structural MRI, functional MRI, diffusion tensor imaging, lesion maps, and functional connectivity data. A lesion analysis module 1304 identifies damaged language circuits and residual functional pathways. A montage optimization module 1306 computes optimal electrode placement to target residual language networks, taking into account the patient's specific lesion pattern and remaining neural pathways.

The optimized montage drives a multi-channel electrode array 1308 capable of delivering targeted stimulation to the identified residual networks, while a rehabilitation exercise application 1310 presents therapeutic exercises including naming tasks, comprehension activities, and speech production exercises. A patient progress tracking module 1312 tracks recovery metrics and adapts stimulation parameters over time based on the patient's response to treatment. The system provides adaptive parameter updates back to the montage optimization module 1306, creating a closed-loop rehabilitation system. A clinical dashboard 1314 provides a provider interface for monitoring patient treatment and outcomes, and progress reports 1316 are generated for healthcare providers to review patient recovery trajectories.

What is claimed is:

1. A transcranial electrical stimulation headband device for enhancing language learning capabilities, the device comprising:
a. electrodes positioned to target language-related brain regions;
b. a stimulation control module configured to control stimulation parameters;
c. a communication interface enabling connection with language learning applications; and
d. wherein the headband is configured to deliver electrical stimulation to specific brain regions during language learning activities.

2. The headband device of claim 1, further comprising a visual guidance feature that uses a smartphone camera to assist users in positioning the headband.

3. The headband device of claim 1, wherein the electrodes comprise at least one of: saline-soaked sponges, hydrogel interfaces, dry electrode technologies, or arrays of conductive pins designed to make contact through hair.

4. The headband device of claim 3, wherein the arrays of conductive pins include spring-loaded mechanisms allowing individual pins to adjust to the contour of the head while maintaining electrical contact.

5. The headband device of claim 1, further comprising extracephalic electrodes implemented with a wristband design functioning as reference or return electrodes, or used to create current paths targeting deeper brain regions.

6. The headband device of claim 1, further comprising an ear reference tab configured to hook over the pinna of the user's ear, providing a consistent anatomical reference point for repeatable anterior-posterior positioning of the headband across sessions.

7. A system for enhancing language learning through targeted transcranial electrical stimulation, the system comprising:
a. a transcranial electrical stimulation headband with electrodes positioned to target language-related brain regions;
b. a language learning application utilizing spaced repetition algorithms;
c. a communication interface between the headband and application enabling synchronization of stimulation with learning activities; and
d. wherein the application triggers the headband to deliver electrical stimulation to specific brain regions when language learning content is presented for learning or review.

8. The system of claim 7, wherein the system is configured to divide language learning content into subsets that receive different stimulation parameters to enable comparative efficacy testing.

9. The system of claim 8, wherein the different stimulation parameters include variations in current intensity, waveform, target brain region, or duration.

10. The system of claim 7, wherein the system includes a sham stimulation mode that delivers current only briefly at the beginning and end of a session with ramping periods to create the sensation of stimulation without delivering current during the primary learning period.

11. The system of claim 7, wherein stimulation parameters are adjusted based on factors including the phase of learning, user performance, or brain region being targeted.

12. A method for enhancing cognitive functions related to language acquisition, the method comprising:
a. applying transcranial electrical stimulation to a user through electrodes positioned over language-related brain regions;
b. synchronizing the delivery of electrical stimulation with language learning activities; and
c. modulating stimulation parameters based on learning context or user response.

13. The method of claim 12, further comprising:
a. presenting language learning content to the user through a language learning application;
b. triggering electrical stimulation in coordination with the presentation of language learning content;
c. recording performance metrics; and
d. analyzing the performance metrics to assess the effects of stimulation.

14. The method of claim 12, further comprising:
a. applying different stimulation protocols to different sets of language learning material; and
b. comparing user performance between the different stimulation protocols to identify optimal stimulation parameters.

15. The method of claim 12, further comprising adjusting stimulation parameters based on factors including the phase of learning, user performance, or the specific language-related cognitive function being engaged.

16. The method of claim 12, wherein the language learning activities utilize spaced repetition algorithms to optimize the timing of information presentation.

17. The system of claim 7, wherein the language learning content comprises at least one of:
vocabulary items, language instructional videos, pronunciation audio, grammar examples, conversational dialogues, or interactive language exercises.

18. The method of claim 13, wherein the language learning content comprises at least one of:
vocabulary items, language instructional videos, pronunciation audio, grammar examples, conversational dialogues, or interactive language exercises.

19. A system for language rehabilitation therapy comprising:
a. a transcranial electrical stimulation headband with electrodes positioned to target language-related brain regions;
b. a therapeutic application delivering language rehabilitation exercises;
c. a communication interface between the headband and application enabling synchronization of stimulation with therapeutic exercises; and
d. wherein the system is configured to deliver targeted electrical stimulation to specific brain regions during language rehabilitation activities.

20. The system of claim 19, wherein the language rehabilitation exercises are designed for patients with acquired language disorders including at least one of: aphasia, traumatic brain injury, primary progressive aphasia, or other neurological conditions affecting language function.

21. A method for enhancing language rehabilitation in patients with acquired language disorders, the method comprising:
a. applying transcranial electrical stimulation to a patient through electrodes positioned over language-related brain regions;
b. synchronizing the delivery of electrical stimulation with language rehabilitation exercises;
c. modulating stimulation parameters based on the specific language deficit being treated;
d. tracking clinical outcomes and adapting stimulation parameters based on patient progress; and
e. generating progress reports for healthcare providers.

22. A method for characterizing an individual's dose-response relationship during neurostimulation-enhanced learning, the method comprising:
a. assigning stimulation parameters to individual learning items;
b. delivering electrical stimulation at the assigned parameters during presentation of each learning item;
c. recording a learning outcome for each review of each learning item; and
d. characterizing a dose-response relationship between stimulation parameters and learning outcomes from the accumulated item-level observations.

23. The method of claim 22, wherein the stimulation parameters include at least one of: current intensity, waveform type, stimulation frequency, stimulation duration, or electrode montage.

24. The method of claim 22, wherein characterizing the dose-response relationship comprises applying a Gaussian Process model with a probit link function to the binary learning outcomes.

25. The method of claim 22, wherein assigning stimulation parameters comprises selecting parameters using an adaptive optimization algorithm that updates dose assignments based on accumulated observations.

26. A method for temporally synchronizing transcranial electrical stimulation with memory encoding events during learning, the method comprising:
a. presenting learning content in at least two phases, including a recall phase without stimulation and an encoding phase;
b. delivering electrical stimulation during the encoding phase, comprising a controlled onset from zero to a target intensity, sustained delivery at the target intensity, and a controlled offset from the target intensity to zero; and
c. recording the user's learning outcome for the presented item.

27. The method of claim 26, wherein the encoding phase has a duration in the range of approximately 3 to 10 seconds and the controlled onset and offset each have a duration in the range of approximately 0.5 to 2 seconds.

28. A method for filtering learning outcome observations for dose-response analysis in a neurostimulation-enhanced learning system, the method comprising:
a. recording learning outcomes from a learning system that tracks memory state of individual learning items;
b. classifying each learning outcome based on whether the assessment reflects short-term memory or long-term memory consolidation;
c. excluding from dose-response analysis those outcomes classified as reflecting short-term memory; and
d. including in dose-response analysis only those outcomes classified as reflecting long-term memory consolidation.

29. The method of claim 28, wherein the learning system is a spaced repetition system and classification is based on the card review state, wherein reviews in a learning state or relearning state are classified as short-term memory and reviews in a review state after a spaced interval are classified as long-term memory consolidation.

30. A system for verifying transcranial electrical stimulation delivery, the system comprising:
a. a stimulation control module configured to deliver a test stimulation at a predetermined intensity;
b. a current measurement sensor configured to measure actual current flowing through the electrodes;
c. a comparison module configured to evaluate correspondence between commanded current and measured current; and
d. a verification module that authorizes commencement of a learning session upon satisfactory correspondence between commanded and measured current values.

31. The system of claim 30, wherein the test stimulation comprises a current profile with controlled ramp-up, sustained hold, and controlled ramp-down.
